(19) United States
(12) Patent Application Publication
(10) Pub. No.: US 2007/0100306 A1
(43) Pub. Date: May 3, 2007

(54) Release coating containing thermoplastic polymers

(76) Inventors: James P. DiZio, St. Paul, MN (US); Stephen W. Bany, St. Paul, MN (US); Janice R. Manore, Roberts, WI (US); Ricardo H. da Silva, Americana (BR)

Correspondence Address: 3M INNOVATIVE PROPERTIES COMPANY, PO BOX 33427, ST. PAUL, MN, 55133-3427, US

(21) Appl. No.: 11/268,409

(22) Filed: Nov. 3, 2005

(51) Int. Cl. A61F 13/15; C08L 77/00

(52) U.S. Cl. 604/371; 424/448; 525/431

(57) ABSTRACT

A release coating that includes one or more thermoplastic silicone-containing polymers and one or more other thermoplastic polymers different from the silicone-containing polymer. An article containing the release coating, methods of making the release coating and article.

### BACKGROUND OF THE INVENTION

Normally tacky and pressure sensitive adhesive (PSA) materials have been used for well over half a century. Products of this type, which take the form of tapes, labels, and other types of adhesive coated sheets, must be protected from unintended adhesion to other surfaces. Hence, tapes are typically wound into a roll on their own backing and labels are typically laminated to a release sheet to prevent their accidental adhesion to other surfaces and also to prevent their contamination with air-borne dust and other contaminants.

In order to allow a roll of tape to be unwound without the undesirable transfer of adhesive to the tape baking, it is customary to provide the tape backing with a low adhesion backsize (LAB). Similarly, the release sheet or liner, to which the adhesive coated label is typically laminated, is supplied with a release coating to permit the easy removal of the liner from the label. This LAB or release coating is expected to reproducibly provide an appropriate level of release to the adhesive of interest, to not deleteriously affect the adhesive, and to be resistant to aging so that the release level remains relatively predictable with time.

Various polymers having low critical surface tension such as silicones, fluorine-containing polymers, and long alkyl chain branched polymers are useful as release coatings (e.g., LABs). Long alkyl chain branched polymers are waxy compounds that can be used to prepare release coatings of medium release value which are especially desirable for PSA tapes. Release coating patents also describe the use of silicone polyureas. Suitable silicone polyurea polymers typically include random blocks of polydimethyl siloxane, soft segments of polyethylene oxide or polypropylene oxide, and hard segments of a low molecular weight diamine/diisocyanate product. These can be water-borne or solvent-borne polymers and are typically applied to nonporous films.

There is a need, however, for other low adhesion backsize coatings that can be applied to backings, such as polypropylene backings.

### BRIEF DESCRIPTION OF THE DRAWINGS

FIG. 1 is a schematic representation of a side view of an adhesive article in a roll construction according to the invention.

FIG. 2 is an enlarged cross-sectional view of an adhesive article in accordance with the invention.

FIG. 3 is a perspective view of a diaper showing a diaper frontal tape of the present invention.

### SUMMARY OF THE INVENTION

Many adhesive articles, particularly fastening tapes in the diaper industry, include a substrate, typically a polypropylene backing with a release coating disposed thereon. Other substrates without adhesives also include release coatings. Such articles are provided by the present invention.

In one embodiment, there is provided an article that includes: a substrate; and a release coating disposed on the substrate, wherein the release coating includes: a thermoplastic silicone-containing polymer having greater than 15 percent by weight (wt-%) silicone segments and less than 65 wt-% silicone segments, based on the total weight of the silicone-containing polymer; and a secondary thermoplastic film-forming polymer that is water dispersible. In this embodiment, the release coating is coated out of water and an optional water-miscible cosolvent. Preferably, the components are selected such that the release coating demonstrates an initial 135° peel force of at least 100 grams per 2.5 cm from a reference fastening test tape that includes a tackified styrene-isoprene-styrene pressure sensitive adhesive having a midblock glass transition temperature (Tg) of 263K and coated at a coating weight of 20-40 g/m² (according to the 135° Peel Force Test described in the Examples Section).

In addition to the silicone segments, the thermoplastic silicone-containing polymer also preferably includes hard segments (preferably, at least 25 wt-% hard segments, based on the total weight of the silicone-containing polymer) and soft segments (preferably, at least 15 wt-% soft segments, based on the total weight of the silicone-containing polymer). Preferably, the thermoplastic silicone-containing polymer includes no more than 45 wt-% hard segments and no more than 45 wt-% soft segments.

Preferably, the hard segments of the thermoplastic silicone-containing polymer include at least 1 wt-% —COOH groups, based on the total weight of the silicone-containing polymer. Preferably, the hard segments of the thermoplastic silicone-containing polymer include no more than 5 wt-% —COOH groups, based on the total weight of the silicone-containing polymer.

Preferably, the secondary thermoplastic film-forming polymer is water dispersible. More preferably, the secondary thermoplastic film-forming polymer is selected from the group consisting of a styrene-acrylic copolymer, ethylene-vinyl chloride-vinylacetate terpolymer, vinylacetate-ethylene copolymer, carboxylated vinylacetate-ethylene copolymer, ethylene vinyl acetate copolymer, ethylene-vinyl chloride copolymer, alkene ketene dimer, vinyl-acetate acrylic copolymer, acrylic polymer, polyvinyl acetate homopolymer, and mixtures thereof.

Typically, silicone-containing polymers known in the art are not readily blended with other thermoplastic polymers in a water-based system. That is, many of the large class of silicone-containing polymers and many other thermoplastic polymers do not readily form a water-based system (i.e., water and optionally one or more water-miscible organic liquids) that can provide effective release coatings. This is significant because the silicone-containing polymers described herein can be expensive, whereas many of secondary thermoplastic polymers with which they can be blended as described herein can be much less costly. Thus, such blends can reduce the cost of the overall composition while still providing the desirable properties of the silicone-containing polymers. It is believed that this is because upon drying a coating of the composition on a substrate, at least a portion of the silicone-containing polymer migrates to the surface of the coating.

In one embodiment, there is provided an article that includes: a substrate; and a release coating disposed on the substrate, wherein the release coating includes a thermoplastic silicone-containing polymer and a secondary thermoplastic film-forming polymer that is water-dispersible. The thermoplastic silicone-containing polymer includes silicone segments, hard segments including —COOH groups, and soft segments. In such silicone-containing polymers, the silicone segments are present in an amount of 20 wt-% to 50 wt-%, based on the total weight of the silicone-containing polymer; the hard segments are present in an amount of 25 wt-% to 45 wt-%, based on the total weight of the silicone-containing polymer; the soft segments are present in an amount of 15 wt-% to 45 wt-%, based on the total weight of the silicone-containing polymer; and the —COOH groups are present in an amount of 1 wt-% to 5 wt-%, based on the total weight of the silicone-containing polymer. The thermoplastic silicone-containing polymer is present (in the release coating) in an amount of 5 wt-% to 99 wt-%, and the secondary thermoplastic film-forming polymer is present (in the release coating) in an amount of 1 wt-% to 95 wt-%, based on the total weight of the thermoplastic silicone-containing polymer and the secondary thermoplastic film-forming polymer. In this embodiment, the release coating is coated out of water and an optional water-miscible organic cosolvent. Preferably, the components are selected such that the resultant release coating demonstrates an initial 135° peel force of at least 100 grams per 2.5 cm from a reference fastening test tape that includes a tackified styrene-isoprene-styrene pressure sensitive adhesive having a midblock Tg of 263K and coated at a coating weight of 20-40 g/m².

In one embodiment, there is provided an article that includes: a diaper tape that includes a polypropylene backing including a first major surface and a second major surface; a pressure sensitive adhesive disposed on the first major surface of the backing; and a low adhesion backsize coating disposed on the second major surface of the backing. In this embodiment, the low adhesion backsize includes a thermoplastic silicone-containing polymer and a secondary thermoplastic film-forming polymer that is water dispersible. The thermoplastic silicone-containing polymer includes silicone segments, hard segments including —COOH groups, and soft segments. In such a silicone-containing polymer, the silicone segments are present in an amount of 20 wt-% to 50 wt-%, based on the total weight of the silicone-containing polymer; the hard segments are present in an amount of 25 wt-% to 45 wt-%, based on the total weight of the silicone-containing polymer; the soft segments are present in an amount of 15 wt-% to 45 wt-%, based on the total weight of the silicone-containing polymer; and the —COOH groups are present in an amount of 1 wt-% to 5 wt-%, based on the total weight of the silicone-containing polymer. The thermoplastic silicone-containing polymer is present (in the release coating) in an amount of 5 wt-% to 99 wt-%, and the secondary thermoplastic film-forming polymer is present (in the release coating) in an amount of 1 wt-% to 95 wt-%, based on the total weight of the thermoplastic silicone-containing polymer and the secondary thermoplastic film-forming polymer. Preferably, the components are selected such that the resultant low adhesion backsize coating demonstrates an initial 135° peel force of at least 100 grams per 2.5 cm from a reference fastening test tape that includes a tackified styrene-isoprene-styrene pressure sensitive adhesive having a midblock Tg of 263K and coated at a coating weight of 20-40 g/m².

In certain embodiments, articles of the present invention can have a pressure sensitive adhesive disposed thereon (e.g., on a surface opposite the surface on which the release coating is disposed). Preferably, the pressure sensitive adhesive includes a styrenic block copolymer rubber (e.g., a styrene-isoprene-styrene block copolymer rubber).

In one embodiment, there is provided a method of making an article, the method including: providing a substrate; applying an aqueous dispersion including a blend of a thermoplastic silicone-containing polymer and a secondary thermoplastic film-forming polymer to a surface of the substrate; wherein the thermoplastic silicone-containing polymer includes greater than 15 wt-% and less than 65 wt-% silicone segments, based on the total weight of the silicone-containing polymer; wherein the secondary polymer is water dispersible; and wherein the aqueous dispersion includes a water-miscible cosolvent. The method further includes drying the aqueous dispersion to form a release coating disposed on the substrate. Preferably, the release coating demonstrates an initial 135° peel force of at least 100 grams per 2.5 cm from a reference fastening test tape that includes a tackified styrene-isoprene-styrene pressure sensitive adhesive having a midblock Tg of 263K and coated at a coating weight of 20-40 g/m².

In one embodiment, there is provided a method of making an article, the method including: providing a backing having a first major surface and a second major surface; applying a pressure sensitive adhesive to the first major surface of the backing; and applying an aqueous dispersion including a blend of a thermoplastic silicone-containing polymer and a secondary thermoplastic film-forming polymer to the second major surface of the backing; wherein the thermoplastic silicone-containing polymer includes greater than 15 wt-% and less than 65 wt-% silicone segments, based on the total weight of the polymer; wherein the secondary polymer is water dispersible; and wherein the aqueous dispersion includes a water-miscible cosolvent; and drying the aqueous dispersion to form a low adhesion backsize coating disposed on the backing. Preferably, the low adhesion backsize demonstrates an initial 135° peel force of at least 100 grams per 2.5 cm from a reference fastening test tape that includes a tackified styrene-isoprene-styrene pressure sensitive adhesive having a midblock Tg of 263K and coated at a coating weight of 20-40 g/m².

As used herein, the terms “comprises” and variations thereof do not have a limiting meaning where these terms appear in the description and claims.

As used herein, “a,” “an,” “the,” “at least one,” and “one or more” are used interchangeably. Thus, for example, a release coating that comprises “a” thermoplastic silicone-containing polymer can be interpreted to mean that the release coating includes “one or more” thermoplastic silicone-containing polymers.

As used herein, “thermoplastic” means a polymer that repeatedly can be softened by heating and hardened by cooling through a temperature range characteristic of the polymer without significant molecular degradation taking place, and in the softened state can be shaped by extrusion; “silicone segments” means diorganosiloxane repeating units of the formula —O—SiR₂—; and film-forming means that a polymer when cast from an aqueous dispersion will coalesce and form a continuous film.

As used herein, “low adhesion backsize” or “LAB” refers to a release coating, preferably a film, that exhibits low adhesion to an adhesive, such as a pressure sensitive adhesive (PSA), so that separation occurs substantially between the adhesive and release coating interface. Release coatings can be used in adhesive tape rolls, where the tape is wound upon itself and usage requires unwinding of the tape roll. Such release coatings are typically referred to as LABs. Release coatings can also be used as a “liner” for other adhesive articles such as labels or medical dressing bandages, where the adhesive article is generally supplied as a sheet-like construction, as opposed to a roll construction.

As used herein, “water-dispersible” means that a polymer forms a stable suspension (i.e., a suspension that does not settle out over time such as in an emulsion) of small particles in water. An “aqueous dispersion” of a composition includes one or more water-dispersible polymers, water, and one or more water-miscible organic liquids (e.g., organic solvents).

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

Also herein, the recitations of numerical ranges by endpoints include all numbers subsumed within that range (e.g., 1 to 5 includes 1, 1.5, 2, 2.75, 3, 3.80, 4, 5, etc.).

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### DETAILED DESCRIPTION OF PREFERRED EMBODIMENTS

The present invention provides articles that include effective release coatings on substrates and optionally adhesives. Such substrates can be used in many adhesive articles such as fastening tapes, bandages, labels, etc. More particularly, the present invention provides an effective low adhesion backsize (LAB) on backings that include a polymeric film and a pressure sensitive adhesive disposed on the polymeric film of the backing. Preferred adhesive articles of the present invention are typically frontal tapes used in the diaper industry (i.e., diaper tapes that provide a fastening/release surface on the front portion of a diaper).

In general, the present invention provides a release coating on a substrate. In certain embodiments, the present invention provides a low adhesion backsize (LAB) on the backing opposite the surface on which a pressure sensitive adhesive (PSA) is disposed. For example, with a backing that includes a polymeric film, the PSA is disposed on the polymeric film on one surface and the LAB is disposed on the opposite surface of the backing.

Suitable pressure sensitive adhesives include a styrenic block copolymer rubber (e.g., a styrene-isoprene-styrene block copolymer rubber). In certain embodiments, the pressure sensitive adhesives include a tackified styrene-isoprene-styrene block copolymer. Preferably, the PSA has a midblock Tg of at least 252K, and more preferably at least 258K. Preferably, the PSA has a midblock Tg of at no greater than 266K, and more preferably no greater than 262K. Preferably, the PSA is coated at a coating weight of at least 16 g/m². Preferably, the PSA is coated at a coating weight of no greater than 25 g/m².

The release coating (or LAB) of the present invention includes at least one thermoplastic silicone-containing polymer, preferably a silicone polyurea polymer, and at least one thermoplastic film-forming polymer different than the silicone-containing polymer (i.e., a secondary thermoplastic film-forming polymer). The following description applies to both release coatings and LABs, which is a specific type of release coating.

The release coating can be applied from a water-based system (i.e., a water-borne dispersion). That is, the release coating compositions of the present invention are capable of being dispersed and coated out of a water-based system that includes water. If desired, the aqueous dispersion can also include one or more water-miscible organic liquids (e.g., organic cosolvents). Such organic cosolvents include, for example, 1-methyl-2-pyrrolidinone, propylene glycol, hexyl carbitol, Dowanol PnB, isopropanol, and mixtures thereof. Preferably, if used, the cosolvent is used in an amount of at least ⅓ the weight of the solids in the composition, more preferably, at least ½ the weight of the solids in the composition, and even more preferably, at least 1/1.5 the weight of the solids in the composition. Preferably, if used, the cosolvent is used in an amount of no greater than 3× the weight of the solids in the composition, more preferably, no greater than 2× the weight of the solids in the composition, and even more preferably, no greater than 1.5× the weight of the solids in the composition.

Suitable thermoplastic silicone-containing polymers for use in the present invention include greater than 15 wt-% and less than 65 wt-% silicone-segments, based on the total weight of the silicone-containing polymer. Such thermoplastic silicone-containing polymers preferably also include hard segments and soft segments. Preferably, suitable thermoplastic silicone-containing polymers include at least 25 wt-% hard segments, based on the total weight of the silicone-containing polymer. Preferably, suitable thermoplastic silicone-containing polymers include at least 15 wt-% soft segments, based on the total weight of the silicone-containing polymer. Preferably, suitable thermoplastic silicone-containing polymers include no more than 45 wt-% hard segments. Preferably, suitable thermoplastic silicone-containing polymers include no more than 45 wt-% soft segments. Preferably, suitable thermoplastic silicone-containing polymers include at least 1 wt-% carboxylic acid groups (—COOH groups), based on the total weight of the silicone-containing polymer. Such acid groups are typically incorporated within the hard segments. Preferably, suitable thermoplastic silicone-containing polymers include no more than 5 wt-% —COOH groups.

Suitable secondary thermoplastic polymers for use in the present invention are film-forming polymers. Preferably, the secondary thermoplastic film-forming polymer is water dispersible. More preferably, the secondary thermoplastic film-forming polymer is selected from the group consisting of a styrene-acrylic copolymer, ethylene-vinyl chloride-vinylacetate terpolymer, vinylacetate-ethylene copolymer, carboxylated vinylacetate-ethylene copolymer, ethylene vinyl acetate copolymer, ethylene-vinyl chloride copolymer, alkene ketene dimer, vinyl-acetate acrylic copolymer, acrylic polymer, polyvinyl acetate homopolymer, and mixtures thereof.

Surprisingly, it has been discovered that the silicone-containing polymers described herein will blend in a water-based system with the secondary thermoplastic polymers described herein and provide effective release coatings. It is believed that this is because upon drying a coating of the composition on a substrate, at least a portion of the silicone-containing polymer migrates to the surface of the coating.

Preferably, release coatings of the present invention include at least 5 wt-% of the thermoplastic silicone-containing polymer, based on the total weight of the thermoplastic silicone-containing polymer and the secondary thermoplastic film-forming polymer. More preferably, release coatings include at least 10 wt-% of the thermoplastic silicone-containing polymer. Preferably, release coatings include no more than 99 wt-% of the thermoplastic silicone-containing polymer, based on the total weight of the thermoplastic silicone-containing polymer and the secondary thermoplastic film-forming polymer. More preferably, release coatings include no more than 50 wt-% of the thermoplastic silicone-containing polymer. Even more preferably, release coatings include no more than 20 wt-% of the thermoplastic silicone-containing polymer.

Preferably, release coatings of the present invention include at least 1 wt-% of the secondary thermoplastic film-forming polymer, based on the total weight of the thermoplastic silicone-containing polymer and the secondary thermoplastic film-forming polymer. More preferably, release coatings include at least 5 wt-% of the secondary thermoplastic film-forming polymer. Even more preferably, release coatings include at least 50 wt-% of the secondary thermoplastic film-forming polymer. Even more preferably, release coatings include at least 80 wt-% of the secondary thermoplastic film-forming polymer. Preferably, release coatings include no more than 95 wt-% of the secondary thermoplastic film-forming polymer, based on the total weight of the thermoplastic silicone-containing polymer and the secondary thermoplastic film-forming polymer. More preferably, release coatings include no more than 90 wt-% of the secondary thermoplastic film-forming polymer.

Preferably, the thermoplastic silicone-containing polymer and secondary thermoplastic film-forming polymer, and amounts thereof, are selected to provide an initial 135° peel force of at least 100 grams per 2.5 cm from a reference fastening test tape that includes a tackified styrene-isoprene-styrene pressure sensitive adhesive having a midblock Tg of 263K and coated at a coating weight of 20-40 grams per square meter (g/m²) (according to the 135° Peel Force Test described in the Examples Section). More preferably, the thermoplastic silicone-containing polymer and secondary thermoplastic film-forming polymer, and amounts thereof, are selected to provide desirable properties of unwind force, release coat transfer, and abrasion resistance.

In particularly preferred embodiments, an article that includes a release coating of the present invention demonstrates one or more of the following desirable properties: an initial unwind force of at least 50 grams (g) per 2.5 centimeters (cm) (wherein the article includes a pressure sensitive adhesive that including a tackified styrene-isoprene-styrene block copolymer, has a midblock Tg of 258K to 262K, and is coated at a coating weight of 16-25 g/m², according to the Unwind Test described in the Examples Section); little or no transfer of the release coating to a reference fastening test tape such that the reference fastening test tape, after being applied to a release coating of the present invention and removed therefrom, demonstrates a readhesion 135° peel force from polyethylene of at least 400 grams per 2.5 cm (according to the Readhesion Test described in the Examples Section, which demonstrates transfer of the release coating to the tackified styrene-isoprene-styrene pressure sensitive adhesive used in the reference fastening test tape of the 135° Peel Force Test); and/or no more than a 200% increase in an initial 135° peel force from a reference fastening test tape that includes a tackified styrene-isoprene-styrene pressure sensitive adhesive having a midblock Tg of 263K and coated at a coating weight of 20-40 g/m², after the release coating is abraded with 30 passes of cotton duck canvas.

Preferably, an article that includes a release coating of the present invention demonstrates an initial 135° peel force of at least 100 grams per 2.5 cm from a reference fastening test tape that includes a tackified styrene-isoprene-styrene pressure sensitive adhesive having a midblock Tg of 263K and coated at a coating weight of 20-40 g/m² (according to the 135° Peel Force Test described in the Examples Section). More preferably, such articles demonstrate an initial 135° peel force of at least 150 grams per 2.5 cm from a reference fastening test tape that includes a tackified styrene-isoprene-styrene pressure sensitive adhesive having a midblock Tg of 263K and coated at a coating weight of 20-40 g/m². Even more preferably, such articles demonstrate an initial 135° peel force of at least 200 grams per 2.5 cm from a reference fastening test tape that includes a tackified styrene-isoprene-styrene pressure sensitive adhesive having a midblock Tg of 263K and coated at a coating weight of 20-40 g/m². Preferably, such articles demonstrate an initial 135° peel force of no more than 1000 grams per 2.5 cm from a reference fastening test tape that includes a tackified styrene-isoprene-styrene pressure sensitive adhesive having a midblock Tg of 263K and coated at a coating weight of 20-40 g/m². More preferably, such articles demonstrate an initial 135° peel force of no more than 700 grams per 2.5 cm from a reference fastening test tape that includes a tackified styrene-isoprene-styrene pressure sensitive adhesive having a midblock Tg of 263K and coated at a coating weight of 20-40 g/m². Even more preferably, such articles demonstrate an initial 135° peel force of no more than 500 grams per 2.5 cm from a reference fastening test tape that includes a tackified styrene-isoprene-styrene pressure sensitive adhesive having a midblock Tg of 263K and coated at a coating weight of 20-40 g/m².

Preferably, an article that includes a tackified styrene-isoprene-styrene pressure sensitive adhesive (having a midblock Tg of 258K to 262K coated at a coating weight of 16-25 g/m²) and a release coating of the present invention demonstrates an initial unwind force of at least 50 g per 2.5 cm (according to the Unwind Test described in the Examples Section). More preferably, such articles demonstrate an initial unwind force of at least 75 g per 2.5 cm. Even more preferably, such articles demonstrate an initial unwind force of at least 100 g per 2.5 cm. Preferably, such articles demonstrate an initial unwind force of no more than 500 g per 2.5 cm. More preferably, such articles demonstrate an initial unwind force of no more than 300 g per 2.5 cm. Even more preferably, such articles demonstrate an initial unwind force of no more than 250 g per 2.5 cm.

Preferably, a reference fastening test tape (that includes a tackified styrene-isoprene-styrene pressure sensitive adhesive having a midblock Tg of 263K and coated at a coating weight of 20-40 g/m²), after being applied to a release coating of the present invention and removed therefrom, demonstrates a readhesion 135° peel force from polyethylene of at least 400 g per 2.5 cm. This test is carried out according to the Readhesion Test described in the Examples Section, which demonstrates transfer of the release coating to the pressure sensitive adhesive of the reference fastening test tape used in the 135° Peel Force Test discussed above. More preferably, such articles demonstrate a readhesion 135° peel force from polyethylene of at least 500 g per 2.5 cm. Even more preferably, such articles demonstrate a readhesion 135° peel force from polyethylene of at least 600 g per 2.5 cm.

Preferably, an article that includes a release coating of the present invention demonstrates no more than a 200% increase in an initial 135° peel force from a reference fastening test tape that includes a tackified styrene-isoprene-styrene pressure sensitive adhesive having a midblock Tg of 263K and coated at a coating weight of 20-40 g/m², after the release coating is abraded with 30 passes of cotton duck canvas. (according to the Abrasion Test described in the Examples Section). More preferably, such articles demonstrate no more than a 100% increase in an initial 135° peel force from a reference fastening test tape that includes a tackified styrene-isoprene-styrene pressure sensitive adhesive having a midblock Tg of 263K and coated at a coating weight of 20-40 g/m², after the release coating is abraded with 30 passes of cotton duck canvas. Even more preferably, such articles demonstrate no more than a 50% increase in an initial 135° peel force from a reference fastening test tape that includes a tackified styrene-isoprene-styrene pressure sensitive adhesive having a midblock Tg of 263K and coated at a coating weight of 20-40 g/m², after the release coating is abraded with 30 passes of cotton duck canvas.

Preferably, a release coating (e.g., low adhesion backsize) is disposed on the backing at a coating weight of no greater than 2.0 grams per meter squared (g/m²). More preferably, the coating weight is no greater than 1.5 g/m², and most preferred, no greater than 1.2 g/m². Typically, the coating weight is sufficient to provide desirable properties as described herein above, as described in the Examples Section. Preferably, this means that the coating weight is at least 0.125 g/m², and more preferably, at least 0.25 g/m². The coating weight of the release coating was measured with a x-ray fluorescent machine (Oxford 3000, Oxford Inc., Abingdon, England) and is reported in grams/meter².

Thermoplastic Silicone-Containing Polymers

Release coatings of the present invention include thermoplastic silicone-containing polymers. Suitable thermoplastic silicone-containing polymers for use in the present invention include greater than 15 wt-% and less than 65 wt-% silicone-segments, based on the total weight of the silicone-containing polymer. That is, the total weight of the polymer is composed of greater than 15 wt-% and less than 65 wt-% —OSiR₂— segments. Without prior knowledge of the materials used to make the polymer, the composition of polymer segments can be determined through a combination of analytical techniques, such as mass spectrometry and nuclear magnetic resonance spectroscopy.

Preferably, suitable thermoplastic silicone-containing polymers include at least 20 wt-% silicone segments. More preferably, such thermoplastic silicone-containing polymers include at least 25 wt-% silicone segments. Even more preferably, such thermoplastic silicone-containing polymers include at least 30 wt-% silicone segments. Preferably, such thermoplastic silicone-containing polymers include no more than 50 wt-% silicone segments. More preferably, such thermoplastic silicone-containing polymers include no more than 40 wt-% silicone segments. Even more preferably, such thermoplastic silicone-containing polymers include no more than 35 wt-% silicone segments.

Suitable thermoplastic silicone-containing polymers preferably also include hard segments and soft segments. Preferably, suitable thermoplastic silicone-containing polymers include at least 25 wt-% hard segments, based on the total weight of the silicone-containing polymer. More preferably, such thermoplastic silicone-containing polymers include at least 25 wt-% hard segments. Even more preferably, such thermoplastic silicone-containing polymers include at least 30 wt-% hard segments. Preferably, such thermoplastic silicone-containing polymers include no more than 45 wt-% hard segments. More preferably, such thermoplastic silicone-containing polymers include no more than 40 wt-% hard segments. Even more preferably, such thermoplastic silicone-containing polymers include no more than 35 wt-% hard segments.

Preferably, suitable thermoplastic silicone-containing polymers include at least 15 wt-% soft segments, based on the total weight of the silicone-containing polymer. More preferably, such thermoplastic silicone-containing polymers include at least 20 wt-% soft segments. Even more preferably, such thermoplastic silicone-containing polymers include at least 30 wt-% soft segments. Preferably, such thermoplastic silicone-containing polymers include no more than 45 wt-% soft segments. More preferably, such thermoplastic silicone-containing polymers include no more than 40 wt-% soft segments. Even more preferably, such thermoplastic silicone-containing polymers include no more than 35 wt-% soft segments.

Preferably, suitable thermoplastic silicone-containing polymers include at least 1 wt-% —COOH groups, based on the total weight of the silicone-containing polymer. Such —COOH groups are typically incorporated into hard segments of the silicone-containing polymer. More preferably, such thermoplastic silicone-containing polymers include at least 1.5 wt-% —COOH groups. Preferably, suitable thermoplastic silicone-containing polymers include no more than 5 wt-% —COOH groups, based on the total weight of the silicone-containing polymer. More preferably, such thermoplastic silicone-containing polymers include hard segments that include no more than 4 wt-% —COOH groups. Even more preferably, such thermoplastic silicone-containing polymers include hard segments that include no more than 3 wt-% —COOH groups. It will be understood by one of skill in the art that the carboxylic acid groups (—COOH groups) are in this form when in the “dried down” state, but would be in the form of carboxylate ions (—COO⁻ ions) when in a water-based system. The weight percentages listed above for the carboxylic acid groups apply also to the amount of carboxylate ions.

A preferred example of a thermoplastic silicone-containing polymer is a water-borne or water-dispersible silicone polyurea, an example of which is disclosed in EP Pat. No. 0 380 236 B1 (Leir).

For certain water-borne embodiments, the silicone polyurea is an organopolysiloxane-polyurea block copolymer including the following repeating unit (Formula I): 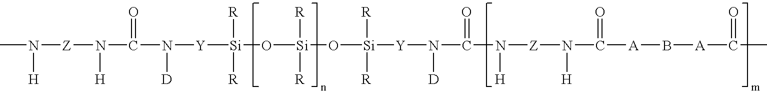
where:
Z is a divalent radical selected from phenylene, alkylene, aralkylene and cycloalkylene;
Y is an alkylene radical of 1 to 10 carbon atoms;
R is at least 50% methyl with the balance of the 100% of all R radicals being selected from a monovalent alkyl radical having from 2 to 12 carbon atoms, a substituted alkyl radical having from 2 to 12 carbon atoms, a vinyl radical, a phenyl radical, and a substituted phenyl radical;
D is selected from hydrogen, an alkyl radical of 1 to 10 carbon atoms, and phenyl;
B is selected from alkylene, aralkylene, cycloalkylene, phenylene, polyethylene oxide, polypropylene oxide, polytetramethylene oxide, polyethylene adipate, polycaprolactone, polybutadiene, mixtures thereof, and a radical which completes a ring structure including A to form a heterocycle, and in the case of the water dispersible forms, contains a sufficient number of in-chain or pendant carboxylic acid groups (or carboxylate ions) to render the block copolymer water-dispersible;
A is selected from the group consisting of —O—, and 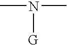
where G is selected from the group consisting of hydrogen, an alkyl radical of 1 to 10 carbon atoms, a phenyl group, and a radical which completes a ring structure including B to form a heterocycle;
“n” is a number which is 10 or larger; and
“m” is a number, other than zero, of up to 25.

The above organopolysiloxane-polyurea block copolymers have the conventional excellent physical properties associated with polysiloxanes of low glass transition temperature, high thermal and oxidative stability, UV resistance, low surface energy and hydrophobicity, good electrical properties and high permeability to many gases, and the additional desirable property of having excellent mechanical and elastomeric properties.

The organosiloxane-polyurethane block copolymers are segmented copolymers of the (AB)ₙ type which can be obtained through a condensation polymerization of a difunctional organopolysiloxane amine (which produces a soft segment) with a diisocyanate (which produces a hard segment) and may include a difunctional chain extender such as a difunctional amine or alcohol, or a mixture thereof.

In the preferred block copolymer Z is selected from hexamethylene, methylene bis-(phenylene), isophorone, tetramethylene, cyclohexylene, and methylene dicyclohexylene and R is methyl.

A method of making the water-borne organopolysiloxane-polyurea block copolymer is described in EP Pat. No. 0 380 236 B1 (Leir). Briefly, the method includes polymerizing a silicone diamine with at least one diisocyanate optionally in the presence of up to 95 weight percent chain extender under an inert atmosphere in a water soluble solvent that has a boiling point of less than 100° C. When the polymerization is completed, water is then added to this mixture to disperse the polymer. The organic solvent is then distilled from the mixture.

The water-borne organopolysiloxane-polyurea block copolymer described in the present invention is composed from the same segments as those described in EP Pat. No. 0 380 236 B1 (Leir). However, the present invention includes an additional method to make the polymer. This additional method is specific for instances when diamino acids are included in the polymer. Briefly, the additional method includes first making an isocyanate end capped solution of oligomers by allowing a silicone diamine and optional chain extenders to react with at least one diisocyanate under an inert atmosphere in a water soluble solvent that has a boiling point of less than 100° C. An aqueous solution of a neutralized diamino acid chain extender is then allowed to react with the oligomeric mix, thus completing the polymer. Solvent is then distilled from the dispersion, yielding substantially water as the dispersing medium.

The silicone diamine preferably has the following general formula (Formula II): 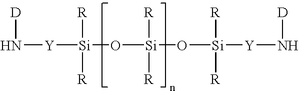
where R, Y, D, and n are as defined in Formula I above. The diisocyanate preferably has a molecular structure represented by OCN-Z-NCO (Formula III) where Z is as defined in Formula I above.

The molar ratio of diamine to diisocyanate is typically maintained in a range of from 1:0.95 to 1:1.05. The chain extender is typically selected from diamines, dihydroxy compounds, and mixtures thereof. When considering water-borne forms of the organopolysiloxane-polyurea block copolymer, at least one of said chain extenders contains at least one group selected from in-chain or pendant carboxylic acid groups, the number of said groups being selected such that, once ionized, said block copolymer preferably having an ionic content of up to 15%; and ionizing said organopolysiloxane-polyurea block copolymer. Typical diamino acid chain extenders useful for the water-borne organopolysiloxane-polyurea block copolymer are 2,5-diaminopentanoic acid, 2,6-diaminopentanoic acid, or diaminobenzoic acid. At least 1.0 wt-% by weight of carboxylate anion, based on the total weight of the silicone-containing polymer, is desired for obtaining a stable dispersion in certain embodiments, with 1-5% being preferred in certain embodiments.

The diisocyanate useful in the reaction can be a phenylene diisocyanate such as toluene diisocyanate or p-phenylene diisocyanate, hexamethylene diisocyanate, aralkylene diisocyanate such as methylene bis-(phenylisocyanate) or tetramethylxylene diisocyanate, or a cycloalkylene diisocyanate such as isophorone diisocyanate, methylene bis(cyclohexyl)diisocyanate, or cyclohexyl diisocyanate.

A method of making the organopolysiloxane diamine represented by Formula II is also described in EP Pat. No. 0 380 236 B1 and U.S. Pat. No. 5,512,650.

The water-dispersible (i.e., water-borne) polymers described in EP Pat. No. 0 380 236 B1 including the process modifications of the present invention use water soluble solvents having lower boiling points than water. Suitable solvents include 2-butanone, tetrahydrofuran, isopropyl alcohol, or mixtures thereof. The acid-containing silicone block copolymer is ionized in solution by deprotonation with stoichiometric amounts of amine or alkaline earth bases such as sodium hydroxide or triethylamine. If desired for a particular application, the amine-containing or carboxylic acid group-containing polymer can be utilized in un-ionized form and coated from solvent.

Secondary Thermoplastic Polymers

Suitable secondary thermoplastic polymers for use in the present invention are film-forming polymers. Preferably, the secondary thermoplastic film-forming polymer is water dispersible.

More preferably, the secondary thermoplastic film-forming polymer is selected from the group consisting of a styrene-acrylic copolymer, ethylene-vinyl chloride-vinylacetate terpolymer, vinylacetate-ethylene copolymer, carboxylated vinylacetate-ethylene copolymer, ethylene vinyl acetate copolymer, ethylene-vinyl chloride copolymer, alkene ketene dimer, vinyl-acetate acrylic copolymer, acrylic polymer, polyvinyl acetate homopolymer, and mixtures thereof.

Optional Additives and Coating Methods

Other compounds, or additives, may be added to release coating compositions including the solvent-borne or water-borne thermoplastic silicone-containing polymer according to the invention to enhance or obtain particular properties. Suitable optional additives are those that preferably do not interfere with the film-forming and release properties of a release coating composition according to the invention. Optional additives are preferably selected from the group of a crosslinker; an antifoam agent; a flow and leveling agent; a colorant (e.g., a dye or a pigment); an adhesion promoter for use with certain substrates; a plasticizer; a thixotropic agent; a rheology modifier; a biocide/anti-fungal agent; a corrosion inhibitor; an antioxidant; a photostabilizer (UV absorber); a surfactant; an emulsifier; an extender (e.g., polymer or polymeric emulsion, thickener, filler); and in the particular case of water-bornes, a film former (e.g., a coalescing organic solvent to assist in film formation); and mixtures thereof. Preferred additives include a surfactant, an antifoam agent, or combinations thereof.

Suitable antifoam agents include, for example, soaps (carboxylates), nitrogenous antifoams such as monoamides, phosphoric acid esters, mineral oil blends, long chain alcohols, fluorosurfactants, silicones, and silica/silicone containing antifoams. Examples of suitable antifoam agents include, for example, those available under the trade designations RG21 (DAG QUIMICA BRAZIL), TA-30 (Taylor Chemical Co. Inc., Lawrenceville, Ga.). Preferably, an antifoam agent, if used, is present in an amount of at least 0.1 wt-%, based on the total weight of the composition. Preferably, an antifoam agent, if used, is present in an amount of no greater than 1 wt-%, based on the total weight of the composition.

Suitable surfactants include, for example, nonionic and ionic types including ethloxylates and propoxylates, fluorinated surfactants, siloxanes, fatty alcohols, fatty acids, fatty esters, alkylamines, carboxylates, sulfonates, and phosphates. Examples of suitable surfactants include, for example, those available under the trade designations SILWET L77 (WITCO BRASIL), ZONYL and MERPOL series (Dupont), FIUORAD series (3M), and STEPANOL series (Stepen). Preferably, a surfactant, if used, is present in an amount of at least 0.005 wt-%, based on the total weight of the composition. Preferably, a surfactant, if used, is present in an amount of no greater than 1 wt-%, based on the total weight of the composition.

The water-borne polymer of the present invention can be coated out of water, or mixtures of water and one or more organic liquids. Release coating compositions provided as aqueous dispersions of the polymer, besides being economical, reduce many problems ordinarily associated with organic solutions and dispersions such as adverse effects of the organic solvent on the surface to be coated, odor, and other environmental concerns during manufacturing.

The desired concentration of the polymer in a release coating composition depends upon the method of coating, upon the desired final coating thickness, and upon the porosity of the substrate. Typically, a release coating composition of the present invention is coated at 5% to 50% solids, and optionally at 10% to 30% solids.

A release coating composition may be applied to a suitable substrate by means of conventional coating techniques such as wire-wound rod, direct gravure, offset gravure, reverse roll, air-knife, and trailing blade coating. The coating can be dried at room temperature, at an elevated temperature, or a combination thereof, provided that the backing material can withstand the elevated temperature. Typically, the elevated temperature is 60° C. to 130° C.

Substrates and Release Coated Materials

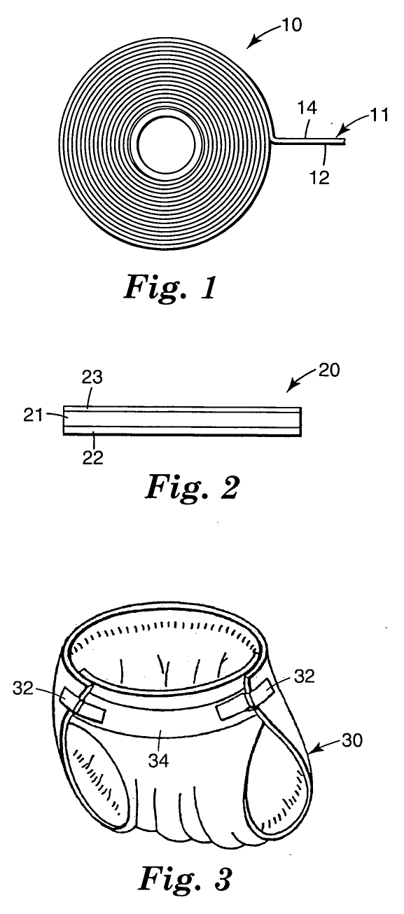

A release coating of the present invention can be used in a variety of formats such as low adhesion backsize (LAB) for pressure-sensitive adhesive (PSA) tapes. For example, as shown in FIG. 1, a roll of tape 10 includes a flexible backing 11, a pressure sensitive adhesive coating on one major surface 12 (i.e., a first major surface) of the backing and a release coating on the opposite major surface 14 (i.e., a second major surface) of the backing. The release coating is formed from the composition described above. The tape is wound into a roll such that the pressure sensitive adhesive contacts the release coating. FIG. 2 is an exploded cross-section of a segment of the tape 10 (FIG. 1).

Referring now to FIG. 2, the tape 20 includes the backing 21, a pressure sensitive adhesive 22, and a release coating (or LAB) 23. The LAB 23 results in a lower specific adhesion toward the pressure sensitive adhesive than does the surface of the backing on which the pressure sensitive adhesive is coated. This permits unwinding of the tape from a roll without offsetting or transfer of the pressure sensitive adhesive from the backing. Another format is a transfer tape including a film of a pressure sensitive adhesive between two release liners, at least one being coated with the release coating composition described above.

A composition of the present invention can be generally used as a release coating for a substrate, which may be a sheet, a fiber, or a shaped object. One preferred type of substrate is that which is used for pressure sensitive adhesive articles, such as tapes, labels, bandages, and the like. The composition may be applied to at least one major surface of suitable flexible or inflexible backing materials before drying is initiated.

Particularly preferred articles including a release coating (or LAB) of the invention are tapes, labels, wound dressings, diaper tapes, and medical grade tapes. For example, one preferred article is a diaper tape that includes a polymeric film, such that the tape is thin, flexible, supple, and conformable. Thus, preferably, the backings are soft, pliable, conformable, and tough. Typically, such backings are strong enough so they don't readily tear when pulled yet soft and flexible enough to be useful in diaper tapes such that they are not uncomfortable to the wearer.

Preferred substrates exhibit a desired combination of properties such as moisture vapor transmission, softness, conformability, yield modulus, texture, appearance, processability, and strength. The particular combination of properties is typically determined by the desired application. For example, for many uses, the substrate will have a low yield modulus and will be of sufficient strength for the desired application and for dispensation in a roll or pad form.

Woven, nonwoven, or knitted materials, or films can be used as the substrate. Useful flexible substrates include woven fabrics formed of threads of synthetic fibers or natural materials such as cotton or blends of these. Alternatively, substrates may be nonwoven fabrics such as carded, spun-bonded, spun-laced, air-laid, and stitch-bonded fabrics of synthetic or natural fibers or blends of these.

Preferably, a substrate includes a material selected from the group consisting of polypropylene (preferably, biaxially oriented polypropylene), polyethylene, polyester, polyimide, and combinations thereof. For diaper tapes, the substrate is typically polypropylene or copolymers of ethylene and propylene.

The substrate is preferably a treated substrate for enhanced adhesion of the release coating. Such treatments include, for example, corona treatment, flame treatment, or chemical treatment.

Pressure sensitive adhesives can be any of a variety of materials known and are generally applied to a backing material. Generally, pressure sensitive adhesives are used in tapes wherein a tape includes a backing (or substrate) and a pressure sensitive adhesive. A pressure sensitive adhesive adheres with no more than applied finger pressure and can be permanently tacky. Pressure sensitive adhesives can be used with primers, tackifiers, plasticizers, and the like. The pressure sensitive adhesives are preferably sufficiently tacky in their normal dry state, and have a desired balance of adhesion, cohesion, stretchiness, elasticity and strength for their intended use.

A release coating of the present invention provides an effective release for a wide variety of conventional pressure-sensitive adhesives such as natural rubber-based, acrylic, tackified block copolymer, and other synthetic film-forming elastomeric materials. Preferably, the pressure sensitive adhesive is nonacidic. That is, it does not include acidic components and is not prepared from acidic monomers such as acidic acrylates, although acrylamide monomers can be used if desired. Particularly preferred pressure sensitive adhesives are block copolymers such as those used in diaper tapes (i.e., diaper fastening tabs). Examples of such adhesives are described in U.S. Pat. No. 5,019,071 (Bany et al.), U.S. Pat. No. 5,453,319 (Gobran), and U.S. Pat. No. 5,468,237 (Miller et al.).

A particularly preferred application of the present invention is in a diaper 30 shown in FIG. 3. Referring to FIG. 3, a diaper 30 has a pressure-sensitive adhesive closure consisting of a pressure-sensitive adhesive tape 32 and a rectangle 34 of polyolefin film covering the fastening area of the diaper. This rectangle 34 is typically referred to as a frontal tape, which preferably includes the release coating of the present invention. The end of the tape has been folded over to provide a narrow nonadhesive tab 36 while leaving a pressure-sensitive adhesive area 38. The diaper can be opened and either discarded, or the closure can be refastened by pressing the adhesive-bearing area 38 of the tape against the rectangle 34.

The present invention is illustrated by the following examples. It is to be understood that the particular examples, materials, amounts, and procedures are to be interpreted broadly in accordance with the scope and spirit of the invention as set forth herein. Unless otherwise indicated, all parts and percentages are by weight and all molecular weights are number average (Mn) molecular weight.

### EXAMPLES

The present invention is illustrated by the following examples. It is to be understood that the particular examples, materials, amounts, and procedures are to be interpreted broadly in accordance with the scope and spirit of the invention as set forth herein. Unless otherwise indicated, all parts and percentages are by weight and all molecular weights are number average (Mn) molecular weight.

### Test Methods

Unwind Force

This test measures the force required to unwind a 51 mm (2 inches) roll of pressure sensitive adhesive (PSA) tape. The unwind force values were determined according to a variation of ASTM D 3811. The tape samples were aged in a constant temperature and humidity room for 24 hours at 21° C. (70° F.) and 50% relative humidity. Three revolutions of tape were removed from a free turning roll, and the roll of tape was centered on the spindle of an unwind apparatus designed to replace the lower jaw on an INSTRON Tester (available from Instron Corporation). The free end of the tape was folded over to form a tab, and the tab was clamped in the upper jaw of the Instron Tester. Approximately 15.2 cm (6 inches) of tape was unwound at a rate of 50.8 cm/min (20 inches/minute), and the average peel value was recorded. Unwind force was also measured on tapes that had been heat aged for 15 days at 49° C. in a forced air convection oven to simulate long term natural aging. The results are reported in grams-force/2.54 cm-width, and represent the average of three independent measurements.

135 Degree Peel Force

The 135 degree peel test was used to measure the amount of force that was required to peel a pressure-sensitive adhesive reference fastening test tape (described below) from the release coated surface of the frontal tape and is used to indicate how well the release coating performs with a given PSA tape.

The frontal tape sample to be tested was securely adhered (release coated side up) to a steel panel measuring two inches×five inches (5.1 cm×12.7 cm) using a double-coated adhesive tape. A one inch (2.5 cm) wide strip of the reference fastening test tape was then adhered to the release coated surface of the frontal tape sample and the test tape was then rolled down with two passes of a 4.5 lb (2 kilogram) rubber roller. The panel was placed into a 135 degree angled fixture that was then placed into the bottom jaw of an INSTRON constant rate tensile tester while the reference fastening test tape was held by the upper jaw. The upper jaw was set in motion at a constant crosshead speed of 12 inches (30.5 cm) per minute while the steel panel was moved so as to keep the adhesive test tape at a 135 degree angle to the panel. The tests were carried out at a constant temperature of 21° C. and 50 percent relative humidity. The force required to remove the reference fastening test tape from the frontal tape was recorded as the release force. The peel force results are reported Table 2 below as 135° Peel Force in grams-force/2.5 centimeter (gm/2.5 cm). The results represent an average of 4 independent measurements. Peel force was also measured on frontal tapes that had been heat aged for 15 days at 49° C. in a forced air convection oven to simulate long term natural aging.

Readhesion

A 135 degree peel test was performed in order to get an indication if any of the release coating had transferred from the release coated side of the frontal tape to the adhesive coated side of the frontal tape after being wound into a roll. A significant decrease in peel force is an indication that some amount of release coating transfer has occurred.

A 13 mil (330 micron) piece of smooth polyethylene film was securely adhered to a steel panel measuring two inches×five inches (5.1 cm×12.7 cm) using double-coated adhesive tape. The same pieces of reference fastening test tape used in the above 135 Degree Peel Force Test, was adhered to the surface of the polyethylene film and then rolled down with two passes of a 2 kilogram rubber roller. The panel was placed into a fixture that was then placed into the bottom jaw of an INSTRON constant rate tensile tester while the reference fastening test tape was held by the upper jaw. The upper jaw was set in motion at a constant crosshead speed of 12 inches (30.5 cm) per minute. The tests were carried out at a constant temperature of 21° C. and 50 percent relative humidity. The force required to remove the reference fastening test tape from the polyethylene film was recorded and reported in Table 2 below as Readhesion in grams-force/2.5 centimeter (gm/2.5 cm). The results represent an average of 4 independent measurements.

Abrasion

The durability of the release coatings of the invention was measured using a Wyzenbeek Precision Wear Test Meter (J.K. Technologies, Rt 6 Box 124, Kankakee, Ill. 60901). The abrading drum was fitted with a cotton duck canvas conforming to Federal Specification CCC-C-419, Type I, No. 6 (available from Test Fabrics Inc., P.O. Box 420, 200 Blackford Ave., Middlesex, N.J. 08846). The tension arm of the test meter was set at 340 grams and the weight bar was set at 150 grams and then locked in place. The frontal tapes from Comparative Examples C3 and C6 and Example 1 were mounted individually into the test meter. The rotating abrading drum was activated for 3, 10, 17 and 30 passes, during which the canvas rubbed or abraded against the release coating side of the frontal tape. The frontal tape was removed from the test meter and then tested using the 135° Peel Force test as described above except only 1 pass of a 4.5 lb (2 kilogram) rubber roller was used to secure the frontal tape to the steel test panel. The results are shown in Table 3 below. Increasing peel force values indicate that the release coating is being abraded off and losing it's functionality as a release coating.

Reference Fastening Test Tape

A pressure-sensitive adhesive reference fastening test tape was prepared by hot melt coating 33.5 grams/meter² of an adhesive consisting of 50% KRATON 1119 (SIS synthetic block copolymer rubber, Kraton Polymers, Inc., Houston, Tex.) and 50% WINGTACK PLUS (synthetic tackifying resin, Goodyear Chemical Co., Akron, Ohio), onto a 94 gram/meter², release coated polypropylene/polyethylene impact copolymer film (7C50, Dow Chemical Co., Midland, Mich.).

Comparative Examples C1-C6 represent commonly used commercially available release materials whose properties herein serve as general targets for the release materials of the invention.

### Comparative Example C1

A frontal tape was prepared by coating a silicone-based release material dispersed in water onto a 25 micron thick biaxially oriented polypropylene (BOPP) film that had been flame treated to have a surface tension of about 40-45 dynes/cm followed by adhesive coating on the non-release side of the film.

The release material was prepared at ambient room temperature (approximately 21° C.) using the following procedure: 25 parts by weight isophorone diisocyanate (IPDI, Bayer Chemicals Pittsburg, Pa.) and 390 parts by weight isopropanol were added to a nitrogen purged reaction vessel. In sequence, and with moderate stirring, 1.33 parts by weight DYTEK-EP (1,3-diaminopentane, Sigma-Aldrich Chemical Co., St. Louis, Mo.), 34.3 parts by weight JEFFAMINE DU700 (Huntsman Performance Chemicals, Houston, Tex.), 29.4 parts by weight silicone diamine (5200 Mn, prepared as in U.S. Pat. No. 5,512,650, Example 38)) and 10 parts by weight isopropanol were added to the resulting solution. The mixture was allowed to react for 10 minutes after which an aqueous solution of 200 parts by weight water, 9.95 parts by weight lysine hydrochloride and 11.8 parts by weight triethylamine were added over a period of 6 minutes with rapid stirring. One hundred parts by weight of water were then added. The mixture was allowed to react for 30 minutes after which 567 parts by weight of liquid were distilled from the solution followed by addition of 267 parts by weight water to yield a 20% solids aqueous dispersion. The silicone content of the final polymer was approximately 30%. The release material dispersion was then further diluted with water to 2.5% solids. This finishes the preparation of the diluted release material dispersion.

1-Methyl-2-pyrrolidinone was added as a cosolvent at an amount equaling that of the solids concentration, which was 2.5% in this case, and then gravure coated onto the BOPP film using a 260 QCH-Quad Channel gravure roll (Consolidated Engravers Inc., Charlotte, N.C.) acting against a rubber roll. The coated web was then passed once through a forced air oven operating at 60° C. to 70° C. to dry the water from the release coating dispersion.

A pressure-sensitive adhesive was then hot melt coated onto the non-release side of the film. The adhesive consisted of a blend of 49.5% styrene-isoprene-styrene block copolymer (KRATON 1107, Kraton Polymers Inc., Houston, Tex.), 49.5% hydrocarbon resin (WINGTACK PLUS, Goodyear Chemicals Inc., Akron, Ohio) and 1% IRGANOX 1076 antioxidant (Ciba Specialty Chemicals, Tarrytown, N.Y.). The adhesive was coated onto the backing using a contact die and a melt temperature of approximately 180° C. The adhesive coating thickness was approximately 20 grams/meter². The resulting frontal tape was then wound upon itself into roll form.

### Comparative Example C2

A frontal tape was prepared as in Comparative Example C1 except the % solids of the final dilution was 1.5%. 1-Methyl-2-pyrrolidinone was added prior to coating at a concentration of 1.5% by weight.

### Comparative Example C3

A frontal tape was prepared as in Comparative Example C1 except a urethane-based release coating was used made by reacting octadecyl isocyanate with a polymer of vinyl alcohol as described in U.S. Pat. No. 2,532,011, and no 1-methyl-2-pyrrolidinone cosolvent was added. A 2.5% solids dispersion in water was used to coat the BOPP film.

### Comparative Example C4

A frontal tape was prepared as in Comparative Example C3 except a 1.5% solids dispersion was used to coat the BOPP film.

### Comparative Example C5

A frontal tape was prepared as in Comparative Example C1 except a silicone/urea-based release coating having a silicone content of 13% was used as described in U.S. Pat. No. 5,356,706, Example 1. A 2.5% solids dispersion with an 1-methyl-2-pyrrolidinone concentration of 2.5% by weight was used to coat the BOPP film.

### Comparative Example C6

A frontal tape was prepared as in Comparative Example C5 except a 1.5% solids dispersion with an 1-methyl-2-pyrrolidinone concentration of 1.5% by weight was used to coat the BOPP film.

### Example 1

A frontal tape was prepared as in Comparative Example C1 except the silicone-based release coating was modified with a water-dispersible thermoplastic polymer.

A 2.5% solids dispersion in water of the silicone polymer in C1 was blended with a 2.5% solids dispersion in water of a styrene-acrylic copolymer (VANCRYL 928, Tg=85° C., Cytec Surface Specialties, Brussels, Belgium) at appropriate ratios with a low agitation stirring apparatus such that when dried the release coating consisted of 20% silicone polymer and 80% styrene-acrylic (SA) copolymer. 1-methyl-2-pyrrolidinone was added as an organic cosolvent at 2.5% by weight. An antifoam agent (RG21, DAG QUIMICA BRAZIL) was added at 0.05% by weight in order to avoid excess of bubbles during the coating process. A modified polysiloxane surfactant (SILWET L77, GE Silicones, Wilton, Conn.) was added at 0.05% in order to decrease the surface tension of the final release coating solution promoting, in this way, a more uniform coating.

The blended dispersion was then coated onto BOPP film and converted into a roll of PSA tape as in C1.

### Example 2

A frontal tape was prepared as in Example 1 except a 1.5% solids dispersion was used to coat the BOPP film. 1-Methyl-2-pyrrolidinone was added as an organic cosolvent at 1.5% by weight. Surfactant and antifoaming agents (i.e., antifoam agents) were not added.

### Example 3

A frontal tape was prepared as in Example 1 except the ratio of silicone polymer to SA copolymer was adjusted to 10:90 on a dry basis. A 2.5% solids dispersion was used to coat the BOPP film. 1-Methyl-2-pyrrolidinone was added as a cosolvent at 2.5% by weight. Surfactant and antifoaming agents were not added.

### Example 4

A frontal tape was prepared as in Example 3 except a 1.5% solids dispersion was used to coat the BOPP film. 1-Methyl-2-pyrrolidinone was added as a cosolvent at 1.5% by weight. Surfactant and antifoaming agents were not added.

### Example 5

A frontal tape was prepared as in Example 1 except an ethylene-vinyl chloride-acetate (EVCA) terpolymer (AIRFLEX 456, Tg=0° C., Air Products and Chemicals Inc., Allentown, Pa.) was used to modify the silicone-based release coating. A 2.5% solids dispersion was used to coat the BOPP film. 1-Methyl-2-pyrrolidinone was added as a cosolvent at 2.5% by weight. Surfactant and antifoaming agents were not added.

### Example 6

A frontal tape was prepared as in Example 5 except a 1.5% solids dispersion was used to coat the BOPP film. 1-Methyl-2-pyrrolidinone was added as a cosolvent at 1.5% by weight. Surfactant and antifoaming agents were not added.

### Example 7

A frontal tape was prepared as in Example 5 except the ratio of silicone polymer to EVCA terpolymer was adjusted to 10:90 on a dry basis. A 2.5% solids dispersion was used to coat the BOPP film. 1-Methyl-2-pyrrolidinone was added as a cosolvent at 2.5% by weight. Surfactant and antifoaming agents were not added.

### Example 8

A frontal tape was prepared as in Example 7 except a 1.5% solids dispersion was used to coat the BOPP film. 1-Methyl-2-pyrrolidinone was added as a cosolvent at 1.5% by weight. Surfactant and antifoaming agents were not added.

### Example 9

A frontal tape was prepared as in Example 1 except a vinylacetate-ethylene (VAE) copolymer (AIRFLEX 144, Tg=0° C., Air Products and Chemicals Inc., Allentown, Pa.) was used to modify the silicone. A 2.5% solids dispersion was used to coat the BOPP film. 1-Methyl-2-pyrrolidinone was added as a cosolvent at 2.5% by weight. Surfactant and antifoaming agents were not added.

### Example 10

A frontal tape was prepared as in Example 9 except a 1.5% solids dispersion was used to coat the BOPP film. 1-Methyl-2-pyrrolidinone was added as a cosolvent at 1.5% by weight. Surfactant and antifoaming agents were not added.

Table 1 below shows that frontal tapes, when made from the modified release coatings of the invention, exhibit unwind forces compared to the unmodified release coatings.

TABLE 1

| | |
|---|---|
| | Unwind Force |
| Release | (gm/2.5 cm) |

| Material | modifier % | % Solids | Initial | Heat Aged |
|---|---|---|---|---|
| C1 | 0 | 2.5 | 89 | 142 |
| C2 | 0 | 1.5 | 102 | 175 |
| C3 | 0 | 2.5 | 141 | 202 |
| C4 | 0 | 1.5 | 158 | 270 |
| C5 | 0 | 2.5 | 138 | 250 |
| C6 | 0 | 1.5 | 155 | 312 |
| 1 | 80 | 2.5 | 106 | 153 |
| 2 | 80 | 1.5 | 113 | 185 |
| 3 | 90 | 2.5 | 128 | 166 |
| 4 | 90 | 1.5 | 179 | 267 |
| 5 | 80 | 2.5 | 104 | 160 |
| 6 | 80 | 1.5 | 218 | 589 |
| 7 | 90 | 2.5 | 129 | 316 |
| 8 | 90 | 1.5 | 585 | 942 |
| 9 | 80 | 2.5 | 178 | 435 |
| 10 | 80 | 1.5 | 213 | 611 |

Table 2 below shows that frontal tapes, when made from the modified release coatings of the invention, exhibit comparable peel forces to the unmodified release coatings.

TABLE 2

| | |
|---|---|
| 135° Peel Force | Readhesion 135° |
| (gm/2.5 cm) | Peel Force |

| Material | Initial | Heat Aged | (gm/2.5 cm) |
|---|---|---|---|
| C1 | 139 | 174 | 710 |
| C2 | 190 | 218 | 655 |
| C3 | 217 | 283 | 811 |
| C4 | 310 | 431 | 723 |
| C5 | 168 | 233 | 606 |
| C6 | 196 | 254 | 622 |
| 1 | 231 | 221 | 731 |
| 2 | 313 | 369 | 673 |
| 3 | 277 | 314 | 706 |
| 4 | 309 | 403 | 647 |
| 5 | 236 | 242 | 644 |
| 6 | 452 | 716 | 609 |
| 7 | 566 | 680 | 659 |
| 8 | 1130 | 1240 | 643 |
| 9 | 469 | 620 | 663 |
| 10 | 481 | 589 | 546 |

Table 3 below shows the dramatic increase in durability (abrasion resistance) of the release coatings of the invention when compared to typical silicone and urethane-based release coatings.

TABLE 3
Peel Force Versus Number of Abrasion Passes

| | 135° Peel Force (gm/2.5 cm) - % Increase | | | | |
|---|---|---|---|---|---|
| Material | 0 Passes | 3 Passes | 10 Passes | 17 Passes | 30 Passes |
| C3 | 152-0% | 168-11% | 241-59% | 411-170% | 502-230% |
| C6 | 131-0% | 218-66% | 390-198% | 560-327% | 683-421% |
| 1 | 155-0% | 157-1% | 163-5% | 197-27% | 218-41% |

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. An article comprising:
a substrate; and
a release coating disposed on the substrate, wherein the release coating comprises:
a thermoplastic silicone-containing polymer comprising greater than 15 wt-% and less than 65 wt-% silicone segments, based on the total weight of the silicone-containing polymer; and
a secondary thermoplastic film-forming polymer that is water dispersible;
wherein the release coating is coated out of a coating composition comprising water and an optional water-miscible organic cosolvent; and
wherein the release coating demonstrates an initial 135° peel force of at least 100 grams per 2.5 cm from a reference fastening test tape that includes a tackified styrene-isoprene-styrene pressure sensitive adhesive having a midblock Tg of 263K and coated at a coating weight of 20-40 g/m².

2. The article of claim 1 wherein the thermoplastic silicone-containing polymer comprises 20 wt-% to 50 wt-% silicone segments.

3. The article of claim 2 wherein the thermoplastic silicone-containing polymer comprises 25 wt-% to 40 wt-% silicone segments.

4. The article of claim 3 wherein the thermoplastic silicone-containing polymer comprises 30 wt-% to 35 wt-% silicone segments.

5. The article of claim 1 wherein the thermoplastic silicone-containing polymer comprises at least 25 wt-% hard segments and at least 15 wt-% soft segments, based on the total weight of the silicone-containing polymer.

6. The article of claim 5 wherein the thermoplastic silicone-containing polymer comprises no more than 45 wt-% hard segments and no more than 45 wt-% soft segments.

7. The article of claim 6 wherein the thermoplastic silicone-containing polymer comprises 25 wt-% to 40 wt-% hard segments and 20 wt-% to 40 wt-% soft segments.

8. The article of claim 7 wherein the thermoplastic silicone-containing polymer comprises 30 wt-% to 35 wt-% hard segments and 30 wt-% to 35 wt-% soft segments.

9. The article of claim 1 wherein the thermoplastic silicone-containing polymer comprises at least 1 wt-% —COOH groups, based on the total weight of the silicone-containing polymer.

10. The article of claim 9 wherein the thermoplastic silicone-containing polymer comprises at least 1.5 wt-% —COOH groups.

11. The article of claim 9 wherein the thermoplastic silicone-containing polymer comprises no more than 5 wt-% —COOH groups.

12. The article of claim 11 wherein the thermoplastic silicone-containing polymer comprises no more than 4 wt-% —COOH groups.

13. The article of claim 1 wherein the coating composition comprises a surfactant, antifoam agent, or combinations thereof.

14. The article of claim 1 wherein the release coating is disposed on the backing at a coating weight of no greater than 2.0 g/m².

15. The article of claim 1 wherein the thermoplastic silicone-containing polymer comprises a silicone polyurea.

16. The article of claim 15 wherein the thermoplastic silicone-containing polymer comprises the following repeating unit: 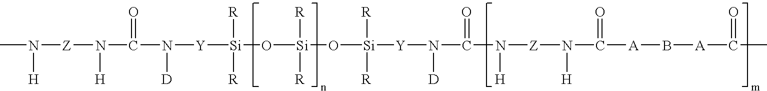
where:
Z is a divalent radical selected from phenylene, alkylene, aralkylene and cycloalkylene;
Y is an alkylene radical of 1 to 10 carbon atoms;
R is at least 50% methyl with the balance of the 100% of all R radicals being selected from a monovalent alkyl radical having from 2 to 12 carbon atoms, a substituted alkyl radical having from 2 to 12 carbon atoms, a vinyl radical, a phenyl radical, and a substituted phenyl radical;
D is selected from hydrogen, an alkyl radical of 1 to 10 carbon atoms, and phenyl;
B is selected from alkylene, aralkylene, cycloalkylene, phenylene, polyethylene oxide, polypropylene oxide, polytetramethylene oxide, polyethylene adipate, polycaprolactone, polybutadiene, mixtures thereof, and a radical which completes a ring structure including A to form a heterocycle, and in-chain or pendant —COOH groups;
A is selected from the group consisting of —O—, and 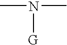
where G is selected from the group consisting of hydrogen, an alkyl radical of 1 to 10 carbon atoms, a phenyl group, and a radical which completes a ring structure including B′ to form a heterocycle;
“n” is a number which is 10 or larger; and
“m” is a number, other than zero, of up to 25.

17. The article of claim 1 wherein the secondary thermoplastic film-forming polymer is selected from the group consisting of a styrene-acrylic copolymer, ethylene-vinyl chloride-vinylacetate terpolymer, vinylacetate-ethylene copolymer, carboxylated vinylacetate-ethylene copolymer, ethylene vinyl acetate copolymer, ethylene-vinyl chloride copolymer, alkene ketene dimer, vinyl-acetate acrylic copolymer, acrylic polymer, polyvinyl acetate homopolymer, and mixtures thereof.

18. The article of claim 1 wherein the optional water-miscible organic cosolvent is selected from the group consisting of 1-methyl-2-pyrrolidinone, propylene glycol, hexyl carbitol, Dowanol PnB, isopropanol, and mixtures thereof.

19. The article of claim 1 wherein the release coating comprises:
at least 5 wt-% of the thermoplastic silicone-containing polymer, based on the total weight of the thermoplastic silicone-containing polymer and the secondary thermoplastic film-forming polymer; and
at least 1 wt-% of the secondary thermoplastic film-forming polymer, based on the total weight of the thermoplastic silicone-containing polymer and the secondary thermoplastic film-forming polymer.

20. The article of claim 19 wherein the release coating comprises:
no more than 99 wt-% of the thermoplastic silicone-containing polymer; and
no more than 95 wt-% of the secondary thermoplastic film-forming polymer.

21. The article of claim 20 wherein the release coating comprises:
5 wt-% to 50 wt-% of the thermoplastic silicone-containing polymer; and
50 wt-% to 95 wt-% of the secondary thermoplastic film-forming polymer.

22. The article of claim 21 wherein the release coating comprises:
10 wt-% to 20 wt-% of the thermoplastic silicone-containing polymer; and
80 wt-% to 90 wt-% of the secondary thermoplastic film-forming polymer.

23. The article of claim 1 which is in the form of a diaper tape.

24. The article of claim 23 wherein the diaper tape is a frontal tape.

25. The article of claim 1 further comprising a pressure sensitive adhesive disposed on the substrate on a surface opposite the surface on which the release coating is disposed, wherein the pressure sensitive adhesive comprises a styrenic block copolymer rubber.

26. The article of claim 25 wherein the pressure sensitive adhesive comprises a tackified styrene-isoprene-styrene block copolymer, has a midblock Tg of 258K to 262K, and is coated at a coating weight of 16-25 g/m², and further wherein the article demonstrates an initial unwind force of at least 50 grams per 2.5 cm.

27. The article of claim 1 wherein the reference fastening test tape, after being applied to the release coating of claim 1 and removed therefrom, demonstrates a readhesion 135° peel force from polyethylene of at least 400 grams per 2.5 cm.

28. The article of claim 1 which demonstrates no more than a 200% increase in an initial 135° peel force from a reference fastening test tape that includes a tackified styrene-isoprene-styrene pressure sensitive adhesive having a midblock Tg of 263K and coated at a coating weight of 20-40 g/m², after the release coating is abraded with 30 passes of cotton duck canvas.

29. An article comprising:
a substrate; and
a release coating disposed on the substrate, wherein the release coating comprises:
a thermoplastic silicone-containing polymer comprising silicone segments, hard segments comprising —COOH groups, and soft segments,
wherein:
the silicone segments are present in an amount of 20 wt-% to 50 wt-%, based on the total weight of the silicone-containing polymer;
the hard segments are present in an amount of 25 wt-% to 45 wt-%, based on the total weight of the silicone-containing polymer;
the soft segments are present in an amount of 15 wt-% to 45 wt-%, based on the total weight of the silicone-containing polymer; and
the —COOH groups are present in an amount of 1 wt-% to 5 wt-%, based on the total weight of the silicone-containing polymer; and
a secondary thermoplastic film-forming polymer that is water dispersible;
wherein the thermoplastic silicone-containing polymer is present in an amount of 5 wt-% to 99 wt-%, based on the total weight of the thermoplastic silicone-containing polymer and the secondary thermoplastic film-forming polymer, and the secondary thermoplastic film-forming polymer is present in an amount of 1 wt-% to 95 wt-%, based on the total weight of the thermoplastic silicone-containing polymer and the secondary thermoplastic film-forming polymer;
wherein the release coating is coated out of a coating composition comprising water and an optional water-miscible organic cosolvent; and
wherein the release coating demonstrates an initial 135° peel force of at least 100 grams per 2.5 cm from a reference fastening test tape that includes a tackified styrene-isoprene-styrene pressure sensitive adhesive having a midblock Tg of 263K and coated at a coating weight of 20-40 g/m².

30. The article of claim 29 wherein the substrate is a corona-treated, flame treated, or chemically treated substrate.

31. The article of claim 29 wherein the substrate comprises a polymer selected from the group consisting of polypropylene, polyethylene, polyester, polyimide, and combinations thereof.

32. The article of claim 29 wherein the secondary thermoplastic film-forming polymer is selected from the group consisting of a styrene-acrylic copolymer, ethylene-vinyl chloride-vinylacetate terpolymer, vinylacetate-ethylene copolymer, carboxylated vinylacetate-ethylene copolymer, ethylene vinyl acetate copolymer, ethylene-vinyl chloride copolymer, alkene ketene dimer, vinyl-acetate acrylic copolymer, acrylic polymer, polyvinyl acetate homopolymer, and mixtures thereof.

33. The article of claim 29 wherein the thermoplastic silicone-containing polymer comprises the following repeating unit: 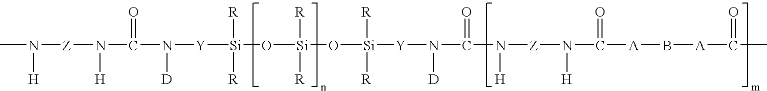
where:
Z is a divalent radical selected from phenylene, alkylene, aralkylene and cycloalkylene;
Y is an alkylene radical of 1 to 10 carbon atoms;
R is at least 50% methyl with the balance of the 100% of all R radicals being selected from a monovalent alkyl radical having from 2 to 12 carbon atoms, a substituted alkyl radical having from 2 to 12 carbon atoms, a vinyl radical, a phenyl radical, and a substituted phenyl radical;
D is selected from hydrogen, an alkyl radical of 1 to 10 carbon atoms, and phenyl;
B is selected from alkylene, aralkylene, cycloalkylene, phenylene, polyethylene oxide, polypropylene oxide, polytetramethylene oxide, polyethylene adipate, polycaprolactone, polybutadiene, mixtures thereof, and a radical which completes a ring structure including A to form a heterocycle, and in-chain or pendant —COOH groups;
A is selected from the group consisting of —O—, and 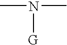
where G is selected from the group consisting of hydrogen, an alkyl radical of 1 to 10 carbon atoms, a phenyl group, and a radical which completes a ring structure including B′ to form a heterocycle;
“n” is a number which is 10 or larger; and
“m” is a number, other than zero, of up to 25.

34. The article of claim 29 further comprising a pressure sensitive adhesive disposed on the substrate on a surface opposite the surface on which the release coating is disposed, wherein the pressure sensitive adhesive comprises a styrenic block copolymer rubber.

34. The article of claim 34 wherein the pressure sensitive adhesive comprises a tackified styrene-isoprene-styrene block copolymer, has a midblock Tg of 258K to 262K, and is coated at a coating weight of 16-25 g/m², and further wherein the article demonstrates an initial unwind force of at least 50 grams per 2.5 cm.

36. The article of claim 29 wherein the reference fastening test tape, after being applied to the release coating of claim 29 and removed therefrom, demonstrates a readhesion 135° peel force from polyethylene of at least 400 grams per 2.5 cm.

37. The article of claim 29 which demonstrates no more than a 200% increase in an initial 135° peel force from a reference fastening test tape that includes a tackified styrene-isoprene-styrene pressure sensitive adhesive having a midblock Tg of 263K and coated at a coating weight of 20-40 g/m², after the release coating is abraded with 30 passes of cotton duck canvas.

38. The article of claim 29 wherein the coating composition comprises a surfactant, antifoam agent, or combinations thereof.

39. A diaper comprising a diaper tape comprising:
a polypropylene backing comprising a first major surface and a second major surface;
a pressure sensitive adhesive disposed on the first major surface of the backing;
a low adhesion backsize coating disposed on the second major surface of the backing, wherein the low adhesion backsize comprises:
a thermoplastic silicone-containing polymer comprising silicone segments, hard segments comprising —COOH groups, and soft segments,
wherein:
the silicone segments are present in an amount of 20 wt-% to 50 wt-%, based on the total weight of the silicone-containing polymer;
the hard segments are present in an amount of 25 wt-% to 45 wt-%, based on the total weight of the silicone-containing polymer;
the soft segments are present in an amount of 15 wt-% to 45 wt-%, based on the total weight of the silicone-containing polymer; and
the —COOH groups are present in an amount of 1 wt-% to 5 wt-%, based on the total weight of the silicone-containing polymer; and
a secondary thermoplastic film-forming polymer that is water dispersible;
wherein the thermoplastic silicone-containing polymer is present in an amount of 5 wt-% to 99 wt-%, based on the total weight of the thermoplastic silicone-containing polymer and the secondary thermoplastic film-forming polymer, and the secondary thermoplastic film-forming polymer is present in an amount of 1 wt-% to 95 wt-%, based on the total weight of the thermoplastic silicone-containing polymer and the secondary thermoplastic film-forming polymer;
wherein the low adhesion backsize is coated out of a coating composition comprising water and an optional water-miscible organic cosolvent; and
wherein the low adhesion backsize coating demonstrates an initial 135° peel force of at least 100 grams per 2.5 cm from a reference fastening test tape that includes a tackified styrene-isoprene-styrene pressure sensitive adhesive having a midblock Tg of 263K and coated at a coating weight of 20-40 g/m².

40. The article of claim 39 wherein the coating composition comprises a surfactant, antifoam agent, or combinations thereof.

41. A method of making an article, the method comprising:
providing a substrate;
applying an aqueous dispersion comprising a blend of a thermoplastic silicone-containing polymer and a secondary thermoplastic film-forming polymer to a surface of the substrate; wherein:
the thermoplastic silicone-containing polymer comprises greater than 15 wt-% and less than 65 wt-% silicone segments, based on the total weight of the silicone-containing polymer;
the secondary thermoplastic film-forming polymer is water dispersible; and
the aqueous dispersion comprises a water-miscible organic cosolvent; and drying the aqueous dispersion to form a release coating disposed on the substrate;
wherein the release coating demonstrates an initial 135° peel force of at least 100 grams per 2.5 cm from a reference fastening test tape that includes a tackified styrene-isoprene-styrene pressure sensitive adhesive having a midblock Tg of 263K and coated at a coating weight of 20-40 g/m².

42. The method of claim 41 wherein the aqueous dispersion comprises a surfactant, antifoam agent, or combinations thereof.

43. A method of making an article, the method comprising:
providing a backing having a first major surface and a second major surface;
applying a pressure sensitive adhesive to the first major surface of the backing; and
applying an aqueous dispersion comprising a blend of a thermoplastic silicone-containing polymer and a secondary thermoplastic film-forming polymer to the second major surface of the backing; wherein:
the thermoplastic silicone-containing polymer comprises greater than 15 wt-% and less than 65 wt-% silicone segments, based on the total weight of the silicone-containing polymer;
the secondary thermoplastic film-forming polymer is water dispersible; and
the aqueous dispersion comprises a water-miscible organic cosolvent; and
drying the aqueous dispersion to form a low adhesion backsize coating disposed on the backing;
wherein the low adhesion backsize coating demonstrates an initial 135° peel force of at least 100 grams per 2.5 cm from a reference fastening test tape that includes a tackified styrene-isoprene-styrene pressure sensitive adhesive having a midblock Tg of 263K and coated at a coating weight of 20-40 g/m².

44. The method of claim 43 wherein the aqueous dispersion comprises a surfactant, antifoam agent, or combinations thereof.
